(19) United States
(12) Patent Application Publication
(10) Pub. No.: US 2019/0032001 A1
(43) Pub. Date: Jan. 31, 2019

(54) CULTURE PEEL PLATE

(71) Applicant: CHARM SCIENCES, INC., Lawrence, MA (US)

(72) Inventor: Robert J. Markovsky, et al., Brentwood, NH (US)

(73) Assignee: Charm Sciences,Inc., Lawrence, MA (US)

(21) Appl. No.: 15/525,110

(22) PCT Filed: Nov. 10, 2015
(86) PCT No.: PCT/US15/59866

§ 371 Date: Oct. 8, 2018

Related U.S. Application Data
(60) Provisional application No. 62/077,566, filed on Nov. 10, 2014.
(60) Provisional application No. 62/079,926, filed on Nov. 14, 2014.

(51) Int. Cl. C12M 1/34; C12M 1/32; C12M 1/00

(52) U.S. Cl. CPC C12M 41/36; C12M 23/12; C12M 41/46; C12M 23/50; C12M 23/38

(57) ABSTRACT

Culture medium devices and systems are shown and described. In one embodiment, a peel plate includes a recessed well, a raised platform, and a removable adhesive cover. In certain examples, the recessed well receives a sample on a dried media culture disc. The removable cover may have a first end secured adjacent to the raised platform and a second removable end removably enclosing the recessed well. The result is a peel plate, assembly, and methods for the enumerating a microorganism, when present, in a sample.

This application claims the benefit of PCT 2015/59866, which claims the benefit of U.S. provisional application No. 62/077566, filed Nov. 10, 2014, and U.S. provisional application No. 62/079926, filed Nov. 14, 2014, all of which are herein incorporated by reference.

### FIELD OF THE TECHNOLOGY

The present disclosure relates generally to analytical testing, and more particularly to improved culture plate devices and methods.

### BACKGROUND

It is desirable to provide rapid, effective detection and identification of various and numerous microorganisms in test samples, such as samples of water, food, such as milk, and body fluids. Microorganisms of interest include all aerobic bacteria and specific bacterial groups, such as coliforms. Other microorganisms of interest include a variety of molds and the like.

Classical methods for culturing various microorganisms for detection and identification thereof include the spread plate method, the pour plate method and the liquid medium method. These methods, however, require preparation by technicians. Such preparation includes mixing, heating and pouring, sterilization of culture medium, apparatus, sample spreading etc. before culture of microorganisms, and inoculation of a test sample into the medium.

Particularly in the food industry, testing is very cost-sensitive. In addition, users might not be laboratory-trained technicians. Tests used must, therefore, be user-friendly and inexpensive without sacrificing accuracy.

Applicants desire an alternative user-friendly culture peel plate device, assembly, and method that is useful, for example, for enumeration, detection, identification, and/or transportation of various microorganisms, without the disadvantages of traditional systems and methods.

### SUMMARY

In accordance with the present disclosure, a culture peel plate is provided for detection, identification, and enumeration of various microorganisms. This disclosure provides an improved peel plate and system that is convenient, efficient, and safe for the user.

One embodiment of the disclosure is an assembly for enumerating a microorganism having a recessed well, a raised platform, a dried media culture disc, and a removable cover seal. Typically, the recessed well receives a sample and the raised platform is adjacent to the recessed well. The dried media culture disc is typically positioned within the recessed well. The removable cover typically includes a first end fixedly secured adjacent to the raised platform, while the opposing second removable end removably encloses the recessed well.

In some examples, the cover's second end includes a peel tab. Further, the removable cover may include an adhesive periphery, for instance on three sides, to removably adhere to an upper face of the plate. The recessed well may be aligned below and parallel to an upper face of the plate. The recessed well may include a grid that is generally visible on the upper face surface and/or lower face surface, for instance for colony counting after the sample has absorbed and diffused throughout the dried media culture disc.

In certain examples, the dried media culture disc supports at least one of (a) an adhesive, (b) a gelling agent, (c) a bacterial nutritive ingredient, (d) a fibrous material, (e) a percent liquid organic solvent, and (f) a cellulosic material. In addition, the plate may include an access indent that is generally opposite the raised platform. The plate may have proximate extensions on opposing sides of the access indent, and the extensions may include an alignment tab.

Another embodiment of the disclosure is a peel plate having a recessed well, a pair of opposing proximate extensions, and a distal raised platform. Typically, the recessed well is spaced between the distal raised platform and the proximate extensions. The recessed well typically has a sunken wall protruding from an upper face. The pair of opposing proximate extensions are typically adjacent to the recessed well, and at least one of the proximate extensions include a proximate tab. The distal raised platform is typically adjacent to the recessed well.

In certain examples, a removable seal cover encloses the recessed well. The cover may include a peel tab, for instance that is removably secured to a proximate end of the plate. Further, the cover may include a self-wicking adhesive periphery removably adhering to an upper face of the plate. The recessed well may be aligned below and parallel to an upper face of the plate and includes a grid. In particular examples, the grid is visible on the upper face surface and/or lower face surface, for instance for colony counting after the sample has absorbed and diffused. The proximate extensions may be spaced between an access indent. The proximate extensions may each include a rounded corner entry to the access indent. A removable seal cover may have a peel tab that is generally aligned along the access indent.

In some examples the distal raised platform spans a length of a diameter of the recessed well. Further, a top perimeter may span around the distal raised platform. The distal platform may include a raised edge extending the platform above the top perimeter.

In particular examples, a culture medium is positioned, i.e. secured or the like, in the recessed well. The culture medium may be a dried media culture disc. The dried media culture disc may comprise a bacterial nutritive ingredient. The dried media culture disc may comprise a growth indicator color-developing agent. For instance, the color-developing agent may be triphenyltetrazolium chloride, 3-(p-iodophenyl)-2-(p-nitrophenyl)-5-phenyl-2H-tetrazolium chloride, 3-(4,5-dimethyl-2-thiazolyl)-2,5-diphenyl-2H-tetrazolium bromide, 5-bromo-3-indolyl-beta.-D-galactoside, bromothymol blue, and neutral red. Further, the dried media culture disc may comprise a plate count agar or plate count agar individual components. In addition, the dried media culture disc may comprise a yeast and mold growth media. The dried media culture disc may comprise a bacterial nutritive ingredient selective medium for growth of indicator organisms. For example, the bacterial nutritive ingredient may comprise selective growth agents for coliform, E. coli, enterbacteriaceae, or pathogens. In addition, the bacterial nutritive ingredient may comprise selective growth agents for salmonella, listeria, or camphlobacter.

In some examples, the dried media culture disc comprises an organic solvent. For instance, the organic solvent may comprise a C1-C5 alcohol. In addition, the organic solvent may comprise 2-propanol. The dried media culture disc may comprise an enhancer. Further, the dried media culture disc may comprise a selective agent.

In particular examples, the sample is a liquid sample. For instance, the liquid sample is a liquid extract selected from the group consisting of a solid, a partial solid, and a combination thereof.

In another embodiment of the disclosure, a peel plate includes a recessed well protruding below an upper face; a raised platform that is adjacent the recessed well and extends in an opposing direction from the recessed well; and an adhesive cover removably enclosing the recessed well.

In certain examples the recessed well includes a grid. For instance, the grid may include a plurality of vertical lines and a plurality of intersecting horizontal lines.

In some examples, the raised platform is positioned on a distal portion of the plate and spans a length of a diameter of the recessed well. Further, the raised platform may include a width to support at least one user's finger, or the like. The raised platform may include a raised edge extending away from the upper face. The plate typically includes a top perimeter around the raised platform. The adhesive cover may include a peel tab removably secured to a proximate end of the plate. An access indent may be aligned opposite the raised platform. The access indent may be positioned between opposing proximate extensions. The proximate extensions may include a proximate tab aligning multiple plates in a layered and/or stacked positioned. Typically, the proximate extensions include a rounded corner entry to the access indent.

Yet another embodiment is a method for enumerating microorganisms on a peel plate having a recessed well, a raised platform, a media culture, and an adhesive cover having a tab. The method typically includes at least one of the following: applying pressure with a user's fingers against the raised platform; lifting the tab vertically upward, thereby exposing the recessed well and maintaining coverage of at least a portion of the cover to the plate; dispensing a sample on the media culture; reapplying the adhesive cover to enclose the recessed well; incubating the peel plate; and enumerating the microorganisms, when present, on the media culture.

The above summary was intended to summarize certain embodiments of the present disclosure. Embodiments will be set forth in more detail in the figures and description of embodiments below. It will be apparent, however, that the description of embodiments is not intended to limit the present inventions, the scope of which should be properly determined by the appended claims.

### BRIEF DESCRIPTION OF THE DRAWINGS

Embodiments of the disclosure will be better understood by a reading of the Description of Embodiments along with a review of the drawings, in which:

FIG. 1 is a top perspective view of one embodiment of an improved culture peel plate according to the disclosure;

FIG. 2 is a bottom perspective view of the culture plate introduced in FIG. 1;

FIG. 3 is a side perspective view of the culture plate introduced in FIG. 1;

FIG. 4 is one example of a diagnostic procedure utilizing a culture peel plate introduced in FIG. 1:

FIG. 5 is another example of a diagnostic procedure utilizing a culture peel plate introduced in FIG. 1:

FIG. 6 is a further example of a diagnostic procedure utilizing a culture peel plate introduced in FIG. 1:

FIG. 7 is yet another example of a diagnostic procedure utilizing a culture peel plate introduced in FIG. 1:

FIG. 8 is one alternative example of a diagnostic procedure utilizing a culture peel plate introduced in FIG. 1: and

FIG. 9 is a top perspective view of one embodiment of a plurality of stacked culture plates according to the disclosure.

### DESCRIPTION OF EMBODIMENTS

In the following description, like reference characters designate like or corresponding parts throughout the several views. Also in the following description, it is to be understood that such terms as “forward,” “rearward,” “left,” “right,” “upwardly,” “downwardly,” and the like are words of convenience and are not to be construed as limiting terms.

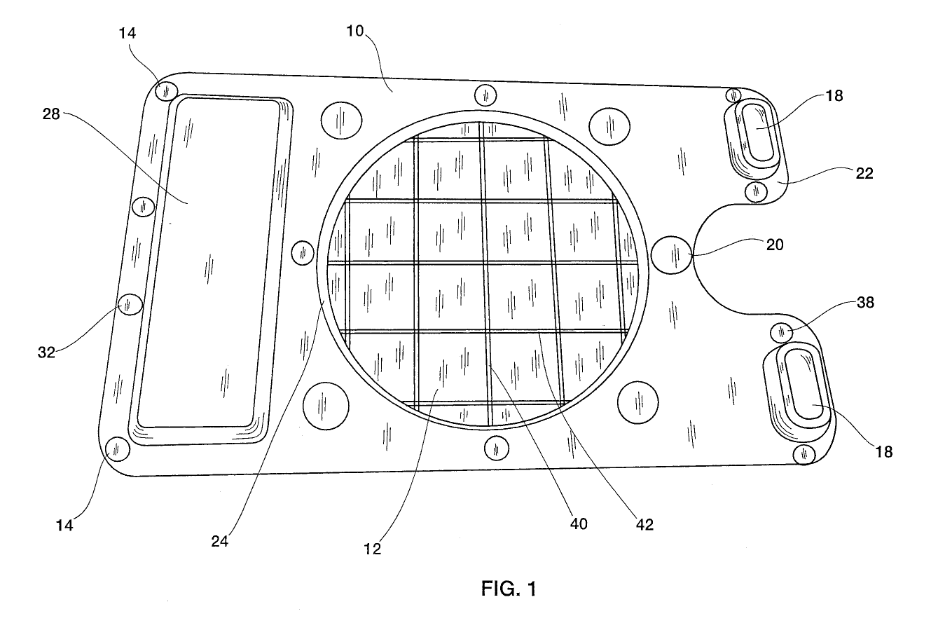

FIG. 1 introduces one example of a culture device peel plate 10 for enumerating and/or detecting a microorganism from a sample. The peel plate 10 is a semi-rigid waterproof plate onto which sample may be applied to enumerate microorganisms and the like. As seen in FIG. 1, one example of the peel plate 10 includes a recessed well 12, a distal raised platform 28, and opposing proximate extensions 22 having proximate tabs 28 to support stacked plates as shown and described herein. The upper face 14 of the plate typically has a top periphery 32 around the raised platform. The recessed well 12 includes a sunken wall 24 below the upper face 14. As shown in FIG. 1, the recessed well may include a grid, for instance having vertical line 40 and intersecting horizontal line 42 components useful for colony counting. In particular examples, the grid is molded into the plate surface. In alternative examples, the grid may be printed in a variety of ways, including inkjet printing, pad printing and the like. Regardless of the grid type, the grid is visible through the generally transparent culture device to the front surface and/or rear surface. The plate 10 is also typically transparent material so as to enable observation from the outside, including any of the grids shown and described herein.

FIG. 1 further shows the proximate end of the peel plate 10 includes an access indent 20 with opposing proximate extensions 22 between rounded corners 38. Typically, the proximate extensions 22 include proximate tabs 18 for alignment, including, but not limited to, layering and stacking plates.

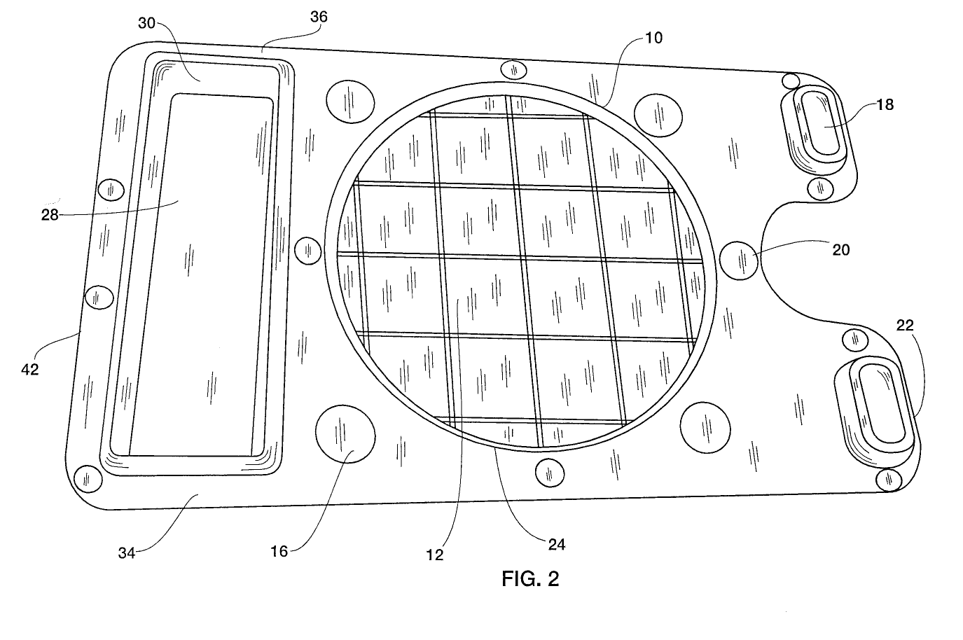

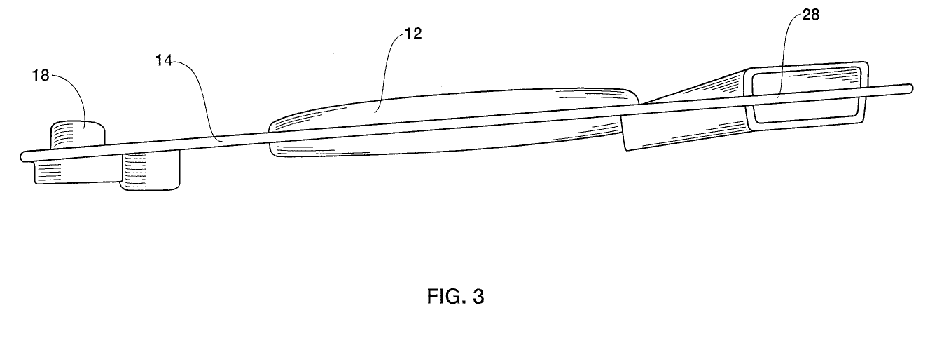

FIGS. 2 and 3 show a bottom and side view, respectively, of one example of a peel plate 10 having a raised edge 30 extending above the lower face 16 to define the raised platform 28. Typically, the peel plate has a distal thickness 42 to support any of the elements and testing procedures shown and described herein.

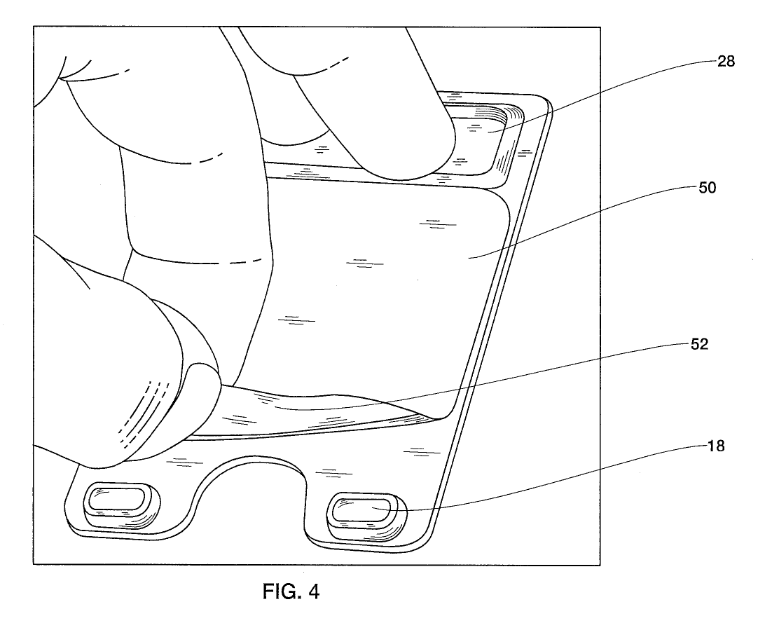

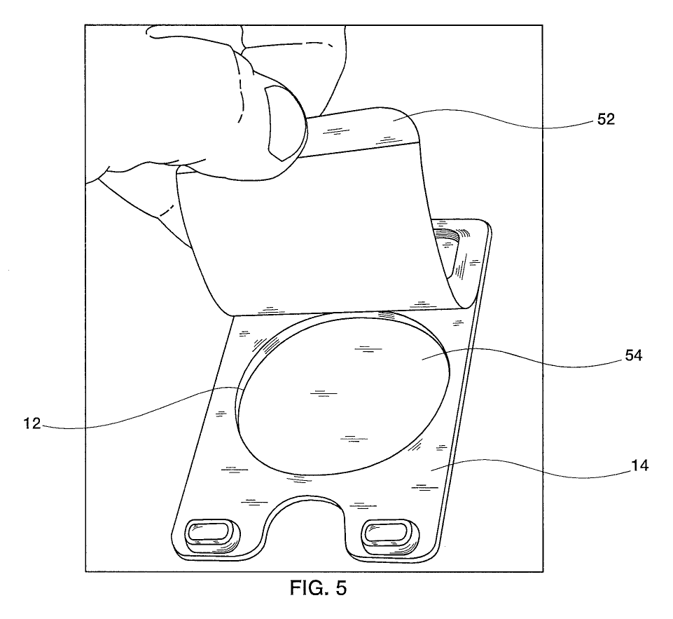

FIG. 4 introduces certain diagnostic steps for enumerating microorganisms, and the like, from a sample. For instance, the peel plate 10 may be placed on a substantially level surface. The peel tab 52 may be lifted concurrently while pressure is applied to the raised platform 28 with the user's fingers, or the like. As next shown in FIG. 5, the tab 52 is lifted vertically upwards and away to expose the culture media 54. In particular the culture media is any of the dried media culture disc shown and described herein. As shown in FIG. 5, the dried media culture disc 54 secured in the recessed well 12 is generally exposed, but the rear portion of the cover remains affixed to a portion of the upper face 14 of the peel plate 10. For instance, the cover remains affixed to the distal portion of the peel plate 10.

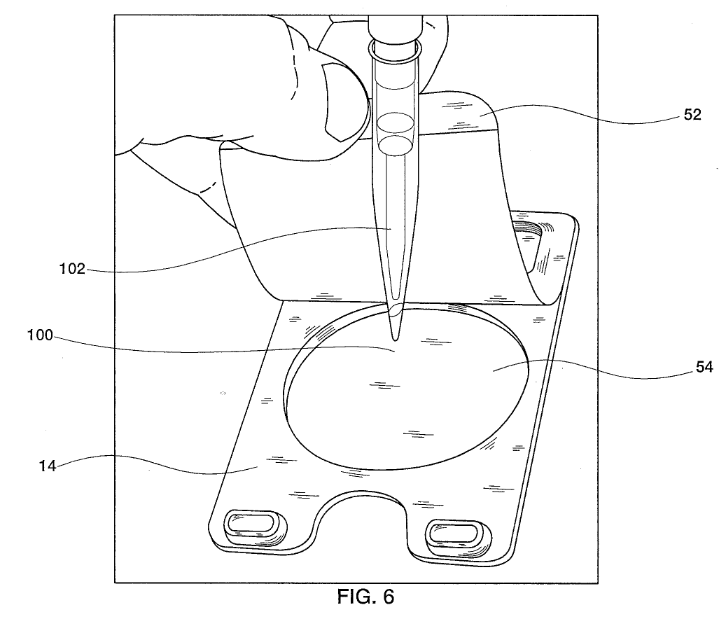

FIG. 6 shows one example of dispensing sample 100 to the recessed well. As shown, the peel plate 10 remains on a flat surface and sample 100 is vertically dispensed to the dried media culture disc 54. In particular examples, the sample is expelled from a pipet 102, or similar laboratory tool, rapidly with substantial force and within about two to about three seconds.

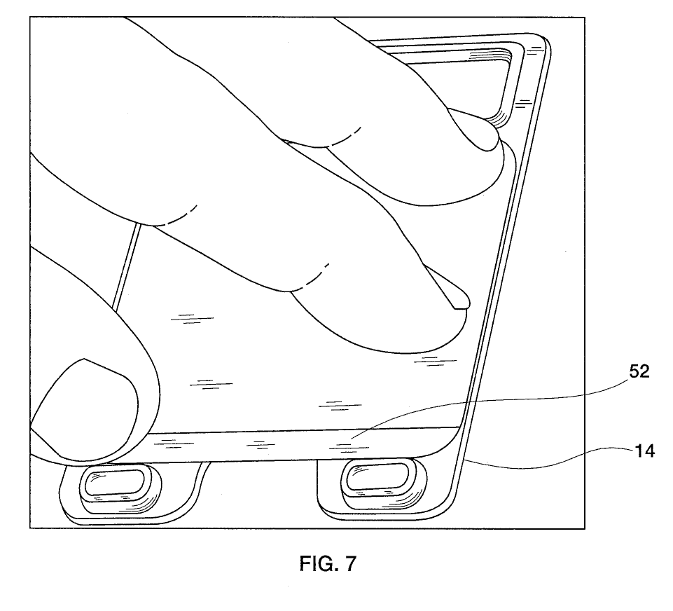

FIG. 7 shows the cover has self-wicking adhesive along surface portions, including but not limited to perimeter portions or edges, to re-apply the adhesive cover to the upper face 14. In particular examples, three edges of the perimeter of the cover include adhesives to self-wick the cover back to the peel plate, for instance by pressing around the edges of the plate to ensure a proper seal. In certain examples, wrinkling the cover may be minimized, or eliminated, by slightly pulling the cover forward while re-applying to the plate.

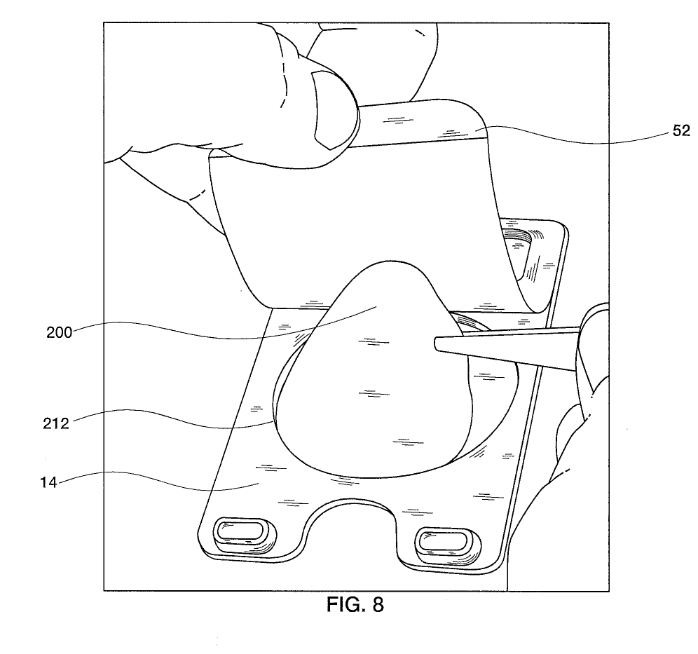

FIG. 8 shows an alternative embodiment for testing filtered water, wherein the sample is filtered through a mixed cellulose filter membrane. In particular examples, an aspirator under partial vacuum, a syringe, or the like filters the sample. In these examples, the adhesive cover is lifted to pre-wet a filter with sterile water, or the like before reapplying the adhesive cover.

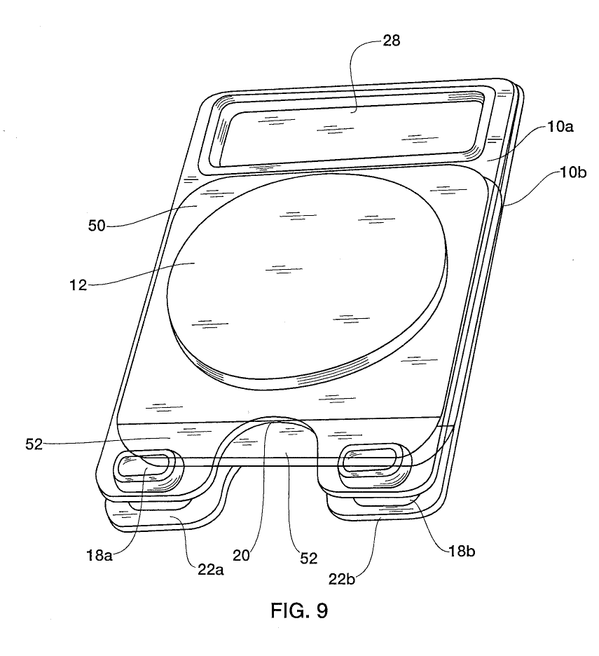

FIG. 9 illustrates one example of layered, or otherwise stacked, peel plates 10a, 10b as shown and described herein. The stacked arrangement may be incubated as understood by those skilled in the art having the benefit of this disclosure. For instance, the stacked plates may be incubated with their respective adhesive covers 50 down and grid sides aligning upward. Applicants have unexpectantly discovered the plates may be stacked by aligning two pillars and the rectangular platform without affecting plate heat transfer.

General good laboratory practices and precautions should be observed for any of the microbial testing shown and described herein. In certain examples, the peel plate's limit of detection includes one colony forming unit per millimeter (CFU/mL) of sample per filtered sample. Generally, at the end of the diagnostic sequence, including after incubation, the plates may be observed for colony growth, for instance through the bottom side of the plates. In certain examples, one spot may represent one CFU. The sum of the spots may be reported as the CFU/mL or CFH/filtered-water-volume pre sample or sample dilution tested. In particular examples, the filtered samples may be viewed on both the bottom and from the top grid side of the filter, for instance after the adhesive cover is removed. Further, the filter membrane may be viewed under illumination and magnification as understood by those skilled in the art having the benefit of this disclosure to count additional colonies while not reducing TTC or producing other pigments. Still further examples include inserting the peel plate into a reader.

In certain examples, a culture medium mixture may be applied to any of the peel plates shown and described herein The culture medium mixture may include an adhesive; a gelling agent; a bacterial nutritive ingredient; a fibrous material; a percent liquid organic solvent; and a cellulosic material. Typically, wherein upon drying or evaporation of the mixture on the device, the mixture forms a liquid absorbent matrix. The matrix may have a uniform distribution of bacterial growth media that is adapted to absorb and evenly diffuse a liquid sample throughout the absorbent matrix.

In some examples, the gelling agent may be xanthan gum, tica gum, locust bean gum, guar gum, carrageenan, alginate, the like and a combination thereof. The adhesive agent may be hydroxypropylcellulose, hydroxylmethylcellulose, solvent soluble cellulosics, the like and a combination thereof. The bacterial nutritive ingredient may comprise EC media. The bacterial nutritive ingredient may comprise plate count agar, plate count agar individual components, the like and a combination thereof. The bacterial nutritive ingredient may comprise yeast and mold growth media. For instance, the bacterial nutritive ingredient may comprise potato dextrose broth.

In particular examples, the bacterial nutritive ingredient may comprise selective medium for growth of indicator organisms. For instance the bacterial nutritive ingredient may comprise growth promoters for bacteria such as coliform, E. coli, enterobacteriaceae, or pathogens. The bacterial nutritive ingredient may comprise growth promoters for pathogens, such as salmonella, listeria, or camphlobacter.

In some examples, the organic solvent comprises ethanol, isopropyl alcohol, the like and a combination thereof. The alcohol percentage may be more than about 50%, including about 60-80%. The organic solvent may contain a percentage of water. The water percentage may be about 1-30%, greater than about 10%, about 15-20% or the like.

The medium may further comprise a growth indicator color-developing agent. In some examples, the color-developing agent includes at least one of the following: triphenyltetrazolium chloride, 3-(p-iodophenyl)-2-(p-nitrophenyl)-5-phenyl-2H-tetrazolium chloride, 3-(4,5-dimethyl-2-thiazolyl)-2,5-diphenyl-2H-tetrazolium bromide, 5-bromo-3-indolyl-beta.-D-galactoside, bromothymol blue, and neutral red. The color-developing agent may include triphenyltetrazolium chloride.

In some examples, the organic solvent comprises a C1-C5 alcohol. In yet other examples, the organic solvent comprises 2-propanol. The medium mixture may include a selective agent. The selective agent may comprise SDS. For instance, the selective agent may comprise bile salts, deoxycholate, sodium citrate or other gram negative selective agents.

Additional medium mixture examples include an enhancer. The enhancer may comprise isopropyl-β-D-thiogalactoside (IPTG) or another similar analogue to lactose. A color enhancer may comprise x-gal.

In particular examples, the fibrous material comprises Rayon, a Rayon derivative, the like or a combination thereof. The fibrous material may include cellulose acetate. Further, the fibrous material may comprise trilobal Rayon. In certain examples, the fibrous material comprises Rayon and wherein the Rayon has a weight of denier filament in the range of about 0.2 mm to about 1.0 mm. For instance the fibrous material comprises Rayon and wherein the Rayon has a denier per filament of about 0.8 to about 3.0.

The selective bacterial nutritive ingredient may comprise components of EC media. The adhesive may comprise polyvinyl pyrrolidone. In other examples the adhesive comprises polyethylene oxide.

In certain examples, the liquid sample is a liquid extract of solid, a partial solid, and a combination thereof.

Another example of the present disclosure includes a colloidal slurry matrix free of a mesh or weave in any of the peel plates shown and described herein. For instance, in a peel plate device for detecting a microorganism, when present, in a liquid sample, a colloidal slurry matrix free of a mesh or weave may include an adhesive; a gelling agent; a bacterial nutritive ingredient; a plurality of Rayon fiber particles; a percent liquid organic solvent; and cellulosic material. Typically, the matrix is a liquid absorbent matrix having a uniform distribution of bacterial growth media that is generally adapted to absorb and evenly diffuse the liquid sample throughout the matrix.

In certain examples, a culture medium and method for detecting microorganisms in a sample, such as a milk sample, a water sample or other food sample, comprising a mixture that can include an adhesive, a gelling agent, a bacterial nutritive ingredient, a fibrous material, a percent liquid organic solvent, and cellulosic material. Upon addition to a waterproof and flat device and being allowed to dry or evaporate the liquid, the mixture forms a bacterial growth media-infused, liquid absorbent and diffusing, matrix that is adhered to the base of the waterproof and flat device.

The gelling agent can be, for example, one or more of tica gum, xanthan gum, locust bean gum, guar gum, carrageenan, or alginate. The adhesive, when present, can be, for example, one or more of Klucel (hydroxypropylcellulose), a solvent soluble cellulosic, for instance carboxymethylcellulose, polyvinyl pyrrolidone and polyethylene oxide. The culture medium bacterial nutritive ingredient can be, for example, EC media or one or more of its individual components thereof or other media and media components known in the art. In other examples, the bacterial nutritive ingredient can be plate count agar or its individual components. The culture medium can also include one or more growth indicator color-developing agents such as triphenyltetrazolium chloride, 3-(p-iodophenyl)-2-(p-nitrophenyl)-5-phenyl-2H-tetrazolium chloride, 3-(4,5-dimethyl-2-thiazolyl)-2,5-diphenyl-2H-tetrazolium bromide, 5-Bromo-4-Chloro-3-Indolyl-B-D-glucuronide; 6-Chloro-3-Indolyl-B-D-galactopyranoside; 5-bromo-3-indolyl-.beta.-D-galactoside, bromothymol blue, and neutral red. The organic solvent can include one or more of ethanol and isopropyl alcohol and can also include, in some aspects, a percentage of water, for example 1-30% water, more than 10% water or in the range of 15-20% water.

In other examples, the solvent can also include one or more alcohols, for example more than 50% or in the range of about 60% to about 80%. Useful alcohols include C1-C5 alcohols such as 2-propanol and ethanol. The waterproof flat plate can be made of, for example, plastic or glass and can be in a typical Petri dish configuration or in a clamshell-like configuration as shown in FIG. 1, and may also include PET plastics, polypropylene, polypropylene and the like. The culture medium can also include an enhancer such as IPTG or another similar analogue to lactose. The fibrous material can include materials such as Rayon or Rayon derivatives or similar materials such as trilobal Rayon, which can be used with a hydrophilic cellulose ester, such as cellulose acetate. The culture medium can also include one or more selective agents such as SDS, antibiotics, deoxycholate, citrate and/or bile salts.

The following experiments demonstrate the efficacy and utility of the present inventions.

### EXAMPLES

### Example 1

### Peel Plates and Petrifilm Raw Milk Analysis

The peel plates shown and discussed herein, described below as TL Peel

Plate; conventional plates, described below as PL Peel Plate; and petrifilm were used during enumerating and detecting aerobic bacteria in milk following Standard Method of Dairy Analysis. The results in Table 1 reflect data generated utilizing the peel plates herein, conventional plates, and petrifilm platforms.

TABLE 1
Aerobic Count Sensitivity

| Dilution | PCA | | Petrifilm | | PL Peel Plate | | TL Peel Plate | |
|---|---|---|---|---|---|---|---|---|
| 10-1 | 126 | 118 | 147 | 137 | 133 | 116 | 123 | 115 |
| 10-2 | 26 | 8 | 27 | 28 | 20 | 19 | 21 | 21 |
| 10-1 | TNTC | TNTC | TNTC | TNTC | TNTC | TNTC | TNTC | TNTC |
| 10-2 | 75 | 71 | 69 | 86 | 56 | 55 | 50 | 73 |
| 10-1 | TNTC | TNTC | TNTC | TNTC | TNTC | TNTC | TNTC | TNTC |
| 10-2 | TNTC | TNTC | TNTC | TNTC | TNTC | TNTC | TNTC | TNTC |
| 10-1 | TNTC | TNTC | TNTC | TNTC | TNTC | TNTC | TNTC | TNTC |
| 10-2 | TNTC | TNTC | TNTC | TNTC | 223 | 230 | 225 | 212 |
| 10-1 | 68 | 74 | 119 | 129 | 125 | 130 | 119 | 109 |
| 10-2 | 3 | 12 | 15 | 17 | 14 | 17 | 28 | 16 |
| 10-1 | 46 | 51 | TNTC | TNTC | TNTC | TNTC | TNTC | TNTC |
| 10-2 | 22 | 16 | 115 | 97 | 78 | 70 | 74 | 70 |
| 10-1 | TNTC | TNTC | TNTC | TNTC | TNTC | TNTC | TNTC | TNTC |
| 10-2 | 56 | 65 | 118 | 119 | 53 | 91 | 78 | 78 |
| 10-1 | TNTC | TNTC | TNTC | TNTC | TNTC | TNTC | TNTC | TNTC |
| 10-2 | TNTC | TNTC | TNTC | TNTC | TNTC | TNTC | TNTC | TNTC |
| 10-1 | 166 | 177 | 161 | 174 | 189 | 209 | 176 | 186 |
| 10-2 | 17 | 14 | 23 | 28 | 21 | 24 | 35 | 29 |
| 10-1 | 25 | 19 | 69 | 47 | 43 | 38 | 32 | 44 |
| 10-2 | 2 | 0 | 5 | 7 | 5 | 2 | 7 | 4 |
| 10-1 | TNTC | TNTC | 191 | 111 | TNTC | TNTC | TNTC | TNTC |
| 10-2 | 54 | 82 | 135 | 180 | 188 | 209 | 194 | 168 |
| 10-1 | TNTC | TNTC | TNTC | TNTC | TNTC | TNTC | TNTC | TNTC |
| 10-2 | TNTC | TNTC | TNTC | TNTC | TNTC | TNTC | TNTC | TNTC |
| 10-1 | 159 | 168 | 122 | 131 | 129 | 157 | 128 | 149 |
| 10-2 | 21 | 15 | 18 | 24 | 27 | 25 | 21 | 31 |
| 10-1 | 100 | 104 | 77 | 65 | 99 | 89 | 82 | 78 |
| 10-2 | 13 | 11 | 11 | 12 | 14 | 15 | 11 | 13 |
| 10-1 | TNTC | TNTC | TNTC | TNTC | TNTC | TNTC | TNTC | TNTC |
| 10-2 | 32 | 28 | TNTC | TNTC | TNTC | TNTC | TNTC | TNTC |
| 10-1 | TNTC | TNTC | TNTC | TNTC | TNTC | TNTC | TNTC | TNTC |
| 10-2 | 175 | 189 | 285 | 274 | 241 | 255 | 248 | 235 |
| 10-1 | TNTC | TNTC | TNTC | TNTC | TNTC | TNTC | TNTC | TNTC |
| 10-2 | TNTC | TNTC | TNTC | 236 | TNTC | TNTC | TNTC | TNTC |
| 10-1 | TNTC | TNTC | TNTC | TNTC | TNTC | TNTC | TNTC | TNTC |
| 10-2 | TNTC | TNTC | TNTC | TNTC | TNTC | TNTC | TE | TE |

The results displayed in Tables 2 and 3 compare aerobic plate count sensitivity (as specified above) with the Standard Method at serial dilutions and 10-100 multipliers.

TABLE 2

| t-test p values | Petrifilm | PL PP-AC | TL PP-AC |
|---|---|---|---|
| from PCA: | 0.08 | 0.11 | 0.19 |
| Result: | No Sig Dif | No Sig Dif | No Sig Dif |
| from Petriflms: | NA | 0.83 | 0.30 |
| Result: | NA | No Sig Dif | No Sig Dif |
| from PL: | NA | NA | 0.31 |
| Result: | NA | NA | No Sig Dif |

TABLE 3
Σ Difference from PCA:

| Petrifilms | PL PP-AC | TL PP-AC |
|---|---|---|
| 0.180 | 0.211 | 0.226 |
| OOS | OOS | OOS |

### Example 2

### Heterotrophic Count Analysis

Those of ordinary skill in the art having the benefit of this disclosure will recognize a variety of sample preparation procedures. For instance, water samples may be tested directly or serially diluted to a countable range (30 to 3000 CFU/mL). Buffering capacity of the method may neutralize any pH variation in water samples. The pH of rehydrated test may be checked with pH paper, for instance to be within a range 6.6-7.2. Further, chlorine disinfected water may be neutralized with thiosulfate before testing. In addition, the water samples may be filtered through a 47 mm, 0.45-μm cellulose acetate filter using a filtration apparatus, for instance under partial vacuum. The filter was applied to the peel plate rehydrated with 1.5 mL sterile reagent water.

The peel plates shown and described herein were used for the enumeration of live heterotrophic bacteria in water following Standard Methods for the Examination of Water and Wastewater.

The peel plate was positioned onto a level surface. The tabs were lifted and pressure was applied with fingers to the raised platform. The tab was then lifted and pulled to remove the adhesive cover vertically upwards and back/away to fully expose the media culture. A portion of the cover adhered to the back edge of the plate. Next, while holding the cover and maintaining the plate on a flat surface, 1.0 mL of sample was vertically dispensed to the center of exposed culture medium. The culture medium contained a substrate 2,3,5-triphenyltetrazolium chloride (TTC) redox-indicator.

A pipet expelled the sample contents rapidly with even force and within two to three seconds to the culture medium. The sample self-wicked to the edges of the medium culture. Next, the adhesive cover was reapplied by pressing around the perimeter edges of the cover to the plate to create a proper seal. Wrinkling was avoided by slightly pulling the cover forward while reapplying the adhesive cover to the plate. The plates were then stacked and aligned in two opposing pillars and incubated cover down, grid side up at 35±0.5° C. for 48 to 72 hours.

Following the incubation period, the plates were observed for red colonies, as viewed through the bottom side of the plate. Each spot represented 1 CFU. The sum of red spots was reported as the CFU/mL or CFU/filtered-water-volume per sample or sample dilution tested.

The filtered samples were viewed on both the bottom and from the top-grid side of filter once the adhesive cover was removed. The filter membrane was additionally viewed under illumination, magnification, and on angle to count additional colonies not reducing TTC or producing other pigment.

For spreading bacteria, a single CFU was scored for each spot and separated color circle. Blended colonies were scored as a single CFU. Next, the CFU/mL and the dilution were multiplied to calculate a CFU/mL or CFU/g sample. Counts of 30 to 3000 CFU/mL (spread plate) and 20-200 CFU per membrane (filter method) were documented was quantitative results, while counts outside that range were considered estimates. Samples results outside the quantitative range were diluted and retested. Unexpectantly, Applicants uncovered the method limit of detection was one colony forming unit per milliliter (CFU/mL) of water sample or 1 CFU per filtered sample.

Numerous characteristics and advantages have been set forth in the foregoing description, together with details of structure and function. Many of the novel features are pointed out in the appended claims. The disclosure, however, is illustrative only, and changes may be made in detail, especially in matters of shape, size, and arrangement of parts, within the principle of the disclosure, to the full extent indicated by the broad general meaning of the terms in which the general claims are expressed. It is further noted that, as used in this application, the singular forms “a,” “an,” and “the” include plural referents unless expressly and unequivocally limited to one referent.

We claim:

What is claimed is:

1. A peel plate for enumerating a microorganism, when present, in a sample, said peel plate comprising:
a. a recessed well having a sunken wall protruding from an upper face;
b. a pair of opposing proximate extensions adjacent said recessed well, wherein at least one of said proximate extensions includes a raised proximate tab adapted to align multiple plates in a layered positioned; and
c. a distal raised platform adjacent said recessed well, and wherein said recessed well being spaced between said distal raised platform and said proximate extensions.

2. The plate of claim 1, further including a removable cover seal adapted to removably enclose said recessed well.

3. The plate of claim 2, wherein said cover seal includes a peel tab removably secured to a proximate end of said plate.

4. The plate of claim 1, wherein said recessed well being aligned below and parallel to an upper face of said plate and includes a grid.

5. The plate of claim 4, wherein said grid being visible for colony counting after said sample has absorbed and diffused.

6. The plate of claim 1, wherein said proximate extensions being spaced between an access indent.

7. The plate of claim 6, wherein said proximate extensions each include a rounded corner entry to said access indent.

8. The plate of claim 1, wherein said distal raised platform spans about a length of a diameter of said recessed well.

9. The plate of claim 1, further including a culture medium secured in said recessed well.

10. The plate of claim 9, wherein said culture medium is a dried media culture disc.

11. The plate of claim 10, wherein said dried media culture disc comprises a bacterial nutritive ingredient.

12. The plate of claim 10, wherein said dried media culture disc comprises a growth indicator color-developing agent.

13. The plate of claim 12, wherein said color-developing agent is selected from the group consisting of triphenyltetrazolium chloride, 3-(p-iodophenyl)-2-(p-nitrophenyl)-5-phenyl-2H-tetrazolium chloride, 3-(4,5-dimethyl-2-thiazolyl)-2,5-diphenyl-2H-tetrazolium bromide, 5-bromo-3-indolyl-beta.-D-galactoside, bromothymol blue, and neutral red.

14. The plate of claim 10, wherein said dried media culture disc comprises a plate count agar or plate count agar individual components.

15. The plate of claim 10, wherein said dried media culture disc comprises a yeast and mold growth media.

16. The plate of claim 10, wherein said dried media culture disc comprises a bacterial nutritive ingredient selective medium for growth of indicator organisms.

17. The plate of claim 16, wherein said bacterial nutritive ingredient comprises selective growth agents for coliform, E. coli, enterbacteriaceae, or pathogens.

18. The plate of claim 16, wherein said bacterial nutritive ingredient comprises selective growth agents for salmonella, listeria, or camphlobacter.

19. The plate of claim 10, wherein said dried media culture disc comprises an organic solvent.

20. The plate of claim 10, wherein said dried media culture disc comprises an enhancer.

21. A peel plate comprising:
a. a recessed well sunken below an upper face;
b. a raised platform adjacent said recessed well and having a width adapted to support at least one user's finger; and
c. an adhesive cover removably enclosing said recessed well.

22. The plate of claim 21, wherein said recessed well includes a grid having a plurality of vertical lines and a plurality of intersecting horizontal lines being visible on said upper face.

23. The plate of claim 21, wherein said raised platform includes a raised edge extending away from said upper face.

24. The plate of claim 21, wherein said adhesive cover includes a peel tab removably secured to a proximate end of said plate.

25. The plate of claim 21, further including an access indent opposite said raised platform.

26. The plate of claim 25, wherein said access indent being positioned between opposing proximate extensions.

27. The plate of claim 26, wherein said proximate extensions include a proximate tab adapted to align multiple plates in a layered positioned.

28. The plate of claim 27, wherein said proximate extensions each include a rounded corner entry to said access indent.

29. An assembly for enumerating a microorganism, said assembly comprising:
a. a recessed well adapted to receive a sample;
b. a raised platform adjacent said recessed well;
c. a dried media culture positioned within said recessed well; and
d. a removable cover having a first end fixedly secured adjacent to said raised platform and a second removable end adapted to removably enclose said recessed well.

30. The assembly of claim 29, wherein said cover's second end includes a peel tab.

31. The assembly of claim 29, wherein said recessed well being aligned below and parallel to an upper face of said plate.

32. The assembly of claim 29, further including an access indent opposite said raised platform.

33. The assembly of claim 32, further including proximate extensions on opposing sides of said access indent, and wherein said extensions include a raised stacking alignment tab.
